(19) United States
(12) Patent Application Publication
(10) Pub. No.: US 2023/0380755 A1
(43) Pub. Date: Nov. 30, 2023

(54) JOINT EVALUATION APPARATUS, METHOD, AND STORAGE MEDIUM

(71) Applicant: OSAKA UNIVERSITY, Osaka (JP)

(72) Inventors: Issei OGASAWARA, Osaka (JP); Ken NAKATA, Osaka (JP)

(73) Assignee: OSAKA UNIVERSITY, Osaka (JP)

(21) Appl. No.: 18/032,169

(22) PCT Filed: Oct. 19, 2021
(86) PCT No.: PCT/JP2021/038532

§ 371 Date: Apr. 15, 2023

(30) Foreign Application Priority Data
Oct. 23, 2020 (JP) 2020-177994

(51) Int. Cl. A61B 5/00; A61B 5/11

(52) U.S. Cl. CPC A61B 5/4528; A61B 5/1121; A61B 2562/0219

(57) ABSTRACT

A joint evaluation apparatus includes an inertial sensor unit that is attached in a vicinity of a joint with joint axes of the joint to connect bones on both sides parallel to a detection axis and detects motion of a bone of a joint axis of which a range of motion of joint movement is limited, among the joint axes, as a waveform signal, a load detection unit that detects a load applied to the joint, a data obtaining unit that, when detecting generation of the load, obtains the waveform signal to be detected by the inertial sensor unit, in a time direction and an intensity direction, and a feature amount calculation unit that analyzes the waveform signal and calculates a feature amount that evaluates movement quality of the joint.

### TECHNICAL FIELD

The present invention relates to techniques of simply and unboundedly evaluating joint movement that possibly leads to injury or disorder, by translational acceleration or angular velocity of each axis of an inertia sensor attached along a joint axis.

### BACKGROUND ART

For example, the eversion of an elbow observed during an acceleration phase of pitching or the eversion and rotation observed in a knee during a turning motion are exercises that cause these joints to deviate from a normal range of motion, which stresses tissues such as ligaments and joint capsules that resist these motions. In addition, repeated impact stress in the longitudinal direction of a lower leg, such as in running, is a source of stress that causes the accumulation of microdamage in bone and periosteum. The main concern in the field of sports medicine has been the establishment of a measurement method and an evaluation method to simply and accurately assess poor kinematic and dynamic features that can be a risk of injury or disorder, the features being observed during these exercises.

Conventional evaluation methods have been evaluated based on the magnitude of joint angles or the magnitude of joint moments and inter-joint force (Non-Patent Literatures 1 and 2). In addition, recently, a method to measure the motion of a knee during landing with a small inertial sensor and evaluate the correlation between a peak value of acceleration data and a peak knee moment value has been proposed (Non-Patent Literature 3). Moreover, Patent Literature 1 proposes a walking movement evaluation system that uses motion capture, estimates a principal component score including changes in knee joint angle and joint moment associated with a walking motion based on floor reaction force data detected during walking, and evaluates the walking motion.

### CITATION LIST

### Patent Literature

[Patent Literature 1] Japanese Patent No. 5315504

### Non-Patent Literature

[Non-Patent Literature 1] Hewett, T., Myer, G., Ford, K., Heidt, R., Colosimo, A., McLean, S., Bogert, A., Paterno, M., Succop, P. (2005). Biomechanical measures of neuromuscular control and valgus loading of the knee predict anterior cruciate ligament injury risk in female athletes: A Prospective Study. The American Journal of Sports Medicine, 33(4), 492-501. https://dx.doi.org/10.1177/0363546504269591

[Non-Patent Literature 2] Kristianslund, E., Faul, O., Bahr, R., Myklebust, G., Krosshaug, T. (2014). Sidestep cutting technique and knee abduction loading: implications for ACL prevention exercises. British Journal of Sports Medicine 48(9), 779 783. https://dx.doi.org/10.1136/bjsports-2012-091370

[Non-Patent Literature 3] Morgan, A., O'Connor, K. (2019). Evaluation of an accelerometer to assess knee mechanics during a drop landing, Journal of Biomechanics 86, 125-131. https://dx.doi.org/10.1016/j.jbiomech.2019.01.055

### SUMMARY OF INVENTION

### Technical Problem

In Patent Literature 1 and Non-Patent Literatures 1 and 2, in order to calculate a variable such as the magnitude of a joint angle, expensive measurement environment such as a three-dimensional motion capture system or a floor reaction force sensor has been required. In addition, the motion capture system has been constrained by a space that is able to be measured, which significantly has limited the type and extent of exercise that is able to be measured. Furthermore, in order to measure movement with these systems, an analysis process of attaching a plurality of (a large number of) reflective markers to a body surface, obtaining these three-dimensional position coordinates, and then calculating the acceleration or a posture matrix is needed, which has required significant offline processing. Due to these limitations, the method has not been suitable for evaluation and feedback immediately after the movement. The method shown in Non-Patent Literature 3 evaluates only a peak value that transiently appears in time series of acceleration data, and has been insufficient for detailed evaluation of a temporal change in the behavior of a lower limb joint.

In view of the foregoing, the present invention proposes a joint evaluation apparatus, and a method thereof, and a storage medium that are capable of focusing on a relationship between Anterior Cruciate Ligament damage (ACL damage) that occurs frequently in sports injuries, and postural sway characteristics, as well as damage or the like to an elbow joint and other parts, and easily and accurately evaluating and forecasting joint damage or the like, based on the behavior of the joint in a direction with a limited range of motion.

### Solution to Problem

A joint evaluation apparatus according to the present invention includes an inertial sensor unit that is attached in a vicinity of a joint with joint axes of the joint to connect bones on both sides parallel to a detection axis and detects motion of a bone of a joint axis of which a range of motion of joint movement is limited, among the joint axes, as a waveform signal, a load detection unit that detects a load applied to the joint, a data obtaining unit that, when detecting generation of the load, obtains the waveform signal to be detected by the inertial sensor unit, in a time direction and an intensity direction, and a feature amount calculation unit that analyzes the waveform signal and calculates a feature amount that evaluates movement quality of the joint.

In addition, a joint evaluation method according to the present invention includes, when a load applied to a joint to connect bones on both sides is detected by a load detection unit, by an inertial sensor unit attached in a vicinity of the joint and having a detection axis parallel to joint axes of the joint, motion of a bone of a joint axis of which a range of motion of joint movement is limited, among the joint axes, as a waveform signal in a time direction and an intensity direction, and analyzing an obtained waveform signal and calculating a feature amount that evaluates movement quality of the joint.

Moreover, the present invention a non-transitory computer readable storage medium storing a program that causes a computer to perform joint evaluation including, when a load applied to a joint to connect bones on both sides is detected by a load detection unit, by an inertial sensor unit attached in a vicinity of the joint and having a detection axis parallel to joint axes of the joint, motion of a bone of a joint axis of which a range of motion of joint movement is limited, among the joint axes, as a waveform signal in a time direction and an intensity direction, and analyzing an obtained waveform signal and calculating a feature amount that evaluates movement quality of the joint.

According to these inventions, the motion of the bone of the joint axis of which the range of motion of the joint movement is limited, among the joint axes, is detected as a waveform signal based on an anatomical basis, by a mounting type inertial sensor unit. The data obtaining unit, when the generation of the load at time of landing to a floor by one-leg drop is detected, for example, obtains the waveform signal to be detected by the inertial sensor unit in the time direction and the intensity direction. The feature amount calculation unit analyzes an obtained waveform signal and calculates the feature amount that evaluates the movement quality of the joint. In this way, by using the mounting type inertial sensor unit, the motion of the bone in the direction of the joint axis of which the range of motion is limited or rotation of the bone about the joint axis of which the range of motion is limited is detected. Then, by obtaining the feature amount, damage, disorder, or the like of such a joint part is able to be easily and accurately evaluated and forecasted, based on the relationship between postural sway, such as left-right or front-back misalignment, torsion, imbalance, or other instabilities, during movement such as drop landing, and a risk of occurrence of damage or disorder. It is to be noted that the inertial sensor unit is not limited to a mode to detect the motion of all or multiple joint axes and may be a detection signal from one target joint axis, for example. In addition, according to a joint part to be examined or a way to apply a load, a joint axis to be detected may be set up appropriately.

### Advantageous Effects of the Disclosure

According to the present invention, behavior of a joint in a direction in which a range of motion is limited is anatomically meaningfully detected, which enables damage, disorder, or the like to be easily and highly accurately evaluated or forecasted.

### BRIEF DESCRIPTION OF DRAWINGS

FIG. 1 is a block diagram showing an embodiment of a joint evaluation apparatus according to the present invention.

FIG. 2 is a view showing a correspondence relationship between a joint axis in a plurality of directions of a joint and a detection axis of an inertia sensor.

FIG. 3A and FIG. 3B are views illustrating a motion of a subject who performs the quality evaluation of joint movement, FIG. 3A is a view showing a state at time of one-leg drop landing, and FIG. 3B is a view showing a postural state immediately after the state.

FIG. 4A is a block diagram of a control unit showing a first embodiment of the joint evaluation apparatus, and FIG. 4B is a block diagram of a control unit showing a second embodiment of the joint evaluation apparatus.

FIG. 5A to FIG. 5D are views showing a detection result of a certain subject, illustrating a first evaluation method, FIG. 5A shows a detection signal by three sensors, FIG. 5B is a histogram of signal intensity in a movable direction (about a Y axis), FIG. 5C is a histogram of signal intensity in an immovable direction (about a Z axis), and FIG. 5D is a histogram of signal intensity in an immovable direction (about an X axis).

FIG. 6A to FIG. 6D are views showing a detection result of another subject, illustrating a first evaluation method, FIG. 6A shows a detection signal by three sensors, FIG. 6B is a histogram of signal intensity in the movable direction (about the Y axis), FIG. 6C is a histogram of signal intensity in the immovable direction (about the Z axis), and FIG. 6D is a histogram of signal intensity in the immovable direction (about the X axis).

FIG. 7 is a flow chart illustrating a procedure of feature amount calculation processing I.

FIG. 8A to FIG. 8G are views illustrating a second evaluation method, FIG. 8A is a view showing a relationship between a knee and a sensor unit, FIG. 8B is a view showing each detection signal (an acceleration signal) of a plurality of subjects, FIG. 8C is a view of a step (a Z scored, variance-covariance matrix (X) value) to create a data set, FIG. 8D shows a step at which a coordinate transformation matrix is calculated from accumulated data, FIG. 8E shows a step to project from a detection signal of a new subject to be evaluated to a principal component analysis space, FIG. 8F shows an example of a principal component space obtained by a learning process of data, and FIG. 8G is a view, in a test process for two new subjects to be evaluated, showing a state in which results are separately projected in and out of an ellipse.

FIG. 9A corresponds to FIG. 8F and FIG. 9B corresponds to FIG. 8G.

FIG. 10 is a flow chart illustrating a procedure of feature amount calculation processing II.

FIG. 11 is a view showing a threshold value (2SD, 3SD) of evaluation that has been set in the principal component space (PC1, PC2).

### DESCRIPTION OF EMBODIMENTS

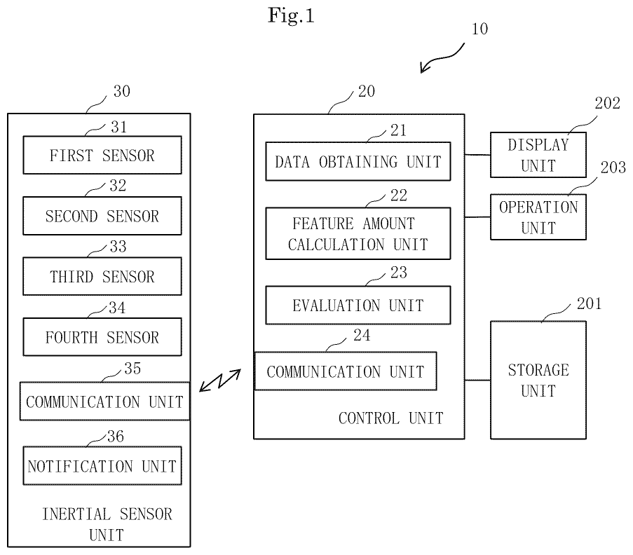

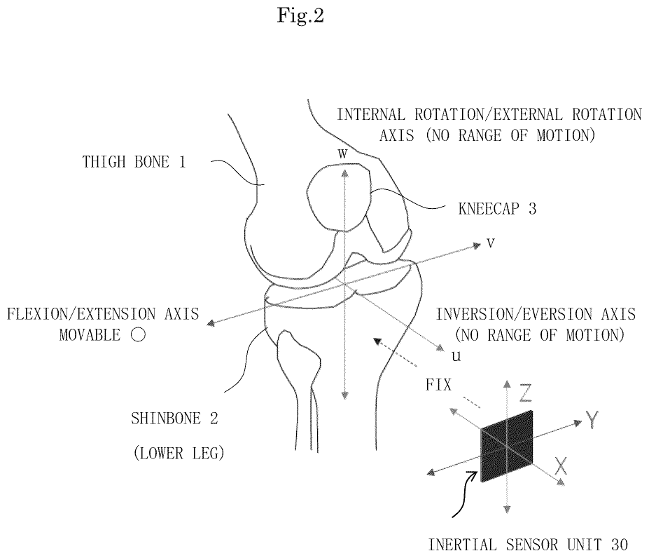

FIG. 1 is a block diagram showing an embodiment of a joint evaluation apparatus according to the present invention, and FIG. 2 is a view showing a correspondence relationship between a joint axis in a plurality of directions of a joint and a detection axis of an inertial sensor unit. In FIG. 1, the joint evaluation apparatus 10 includes a control unit 20 and an inertial sensor unit 30. The inertial sensor unit 30 detects the motion of each joint axis of a joint to be evaluated, for example, a knee joint, and is mounted in a vicinity of the joint to be evaluated. The control unit 20 is typically configured by a computer (a processor), obtains a detection signal from the inertial sensor unit 30, and executes predetermined joint evaluation processing to be described below.

The inertial sensor unit 30 herein includes a first sensor 31 to a third sensor 33, a fourth sensor 34, a communication unit 35, and a notification unit 36 to be provided as necessary. The communication unit 35 sends and receives a signal by wire or wireless with the communication unit 24 near the control unit 20. The notification unit 36 has a pronunciation unit of a beep (beep) sound, for example, and, in a case in which the evaluation results in the control unit 20 is poor, for example, pronunciation is controlled.

The inertial sensor unit 30 is a miniaturized wearable sensor, for example, a disc-shaped member as shown in FIG. 2, and is fixedly mounted on a knee joint part by a fastening tool. The fastening tool may be a string, a band, a hook and loop fastener, or the like, as well as an adhesive material.

The mounting posture on the knee joint is shown in FIG. 2. In FIG. 2, the knee joint part couples a thigh bone 1 and a shinbone (a lower leg) 2 in a vertical direction (a w direction). It is to be noted that a kneecap 3 is on the front side of the knee joint part, the front-back direction of the knee joint part is defined as a u direction, and the left-right direction is defined as a v direction. Herein, the u, v, and w directions are orthogonal to each other.

Of the knee joint part, the lower leg 2 has a range of motion (flexion/extension) about the v axis relative to the thigh bone 1, while the range of motion is limited (no range of motion) in other directions and about the axis (internal rotation/external rotation and inversion/eversion).

In addition, when the X-Y-Z axes are assumed to be orthogonal to each other, the first sensor 31 to be built in the inertial sensor unit 30 detects an angular velocity about the Z axis, and the second sensor 32 detects an angular velocity about the X axis. The third sensor 33 detects the acceleration in the Y-axis direction, and the fourth sensor 34 detects the acceleration in the Z-axis direction. The mounting position of the inertial sensor unit 30, since detecting the motion of the shinbone 2 with respect to the thigh bone 1 with high accuracy, is preferably at the tibial tuberosity being a front side upper portion of the shinbone 2. Furthermore, the inertial sensor unit 30 is mounted on the knee joint part so as to determine the orientation so that the X axis may be parallel to the u axis, the Y axis may be parallel to the v axis, and the Z axis may be parallel to the w axis. As a result, an anatomical meaning is able to be attached to detection data from the first to the third sensors 31 to 33.

It is to be noted that the inertial sensor unit 30 may be of a general-purpose type including all four sensors of the first sensor 31 to the fourth sensor 34 that are built in as a single unit or, as described below, may be of an exclusive type including only a sensor required in each of the first embodiment and the second embodiment.

Referring back to FIG. 1, the control unit 20 is connected to a storage unit 201, a display unit 202, and an operation unit 203. The storage unit 201 includes a memory area in which a control program, and various types of data or the like required for processing are stored, and a work area in which an operation of obtaining detection data, data processing, and data in the middle of the processing are temporarily stored. The display unit 202 performs the check display of operation content and the display of evaluation results. The operation unit 203 performs various types of input instructions for processing, and may employ a touch panel made of a transparent pressure sensitive element stacked on a surface of the display unit 202.

The control unit 20 executes the control program, and thus functions as a data obtaining unit 21, a feature amount calculation unit 22, an evaluation unit 23, and a communication unit 24.

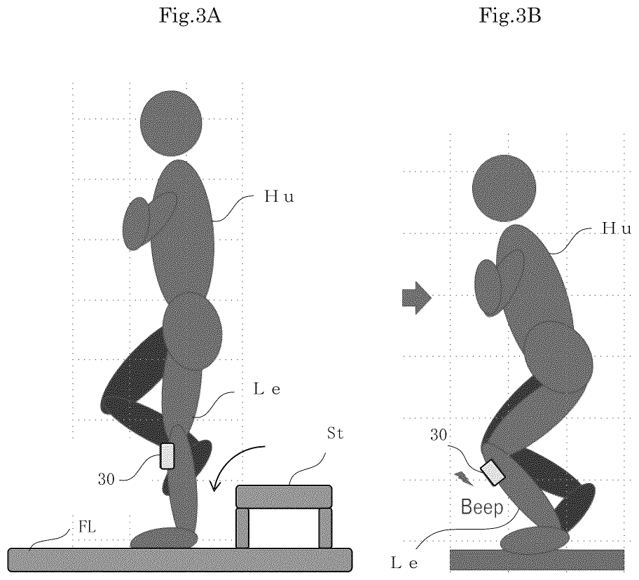

The data obtaining unit 21 samples a detection signal from the first sensor 31 to the fourth sensor 34 with a predetermined period, and obtains the detection signal as a waveform signal for a predetermined time. The data obtaining unit 21, as shown in FIGS. 3A and 3B, starts detection of a signal from a time point when a subject Hu drops from a predetermined height, for example, a 20 cm stand St to a floor FL and lands on a leg Le on a side to be evaluated. The landing timing of drop jump is determined from a change in the acceleration detected by the fourth sensor 34. In other words, the data obtaining unit 21, when detecting that the acceleration in the Z-axis direction from the fourth sensor 34 exceeds a predetermined threshold value, for example, 7G (see FIG. 3A), determines a landing, and starts capturing a detection signal. FIG. 3B shows a postural state of the subject Hu immediately after the state, and the motion of the target knee joint is detected from a landing time point. It is to be noted that the subject tries to maintain the posture at the time of landing for several seconds after landing, for example, about 5 seconds, and the data for a predetermined time out of the time is used for evaluation.

The feature amount calculation unit 22 calculates a feature amount from the detection signal from the first sensor 31 to the third sensor 33. A method of calculating a feature amount includes the first embodiment and the second embodiment that have different the data obtaining processing and the feature amount calculation processing. Each embodiment will be described below with reference to the drawings.

The joint evaluation, while being able to be performed by manually evaluating the feature amount calculated by the feature amount calculation unit 22, may be automatically evaluated. The evaluation unit 23 is provided as necessary, displays the feature amount calculated by the feature amount calculation unit 22, while being associated with a threshold value for good or bad evaluation of the quality of joint movement (manual evaluation), or notifies on a screen whether the feature amount is either inside or outside of the threshold value, or outputs instructions to notify from the notification unit 36.

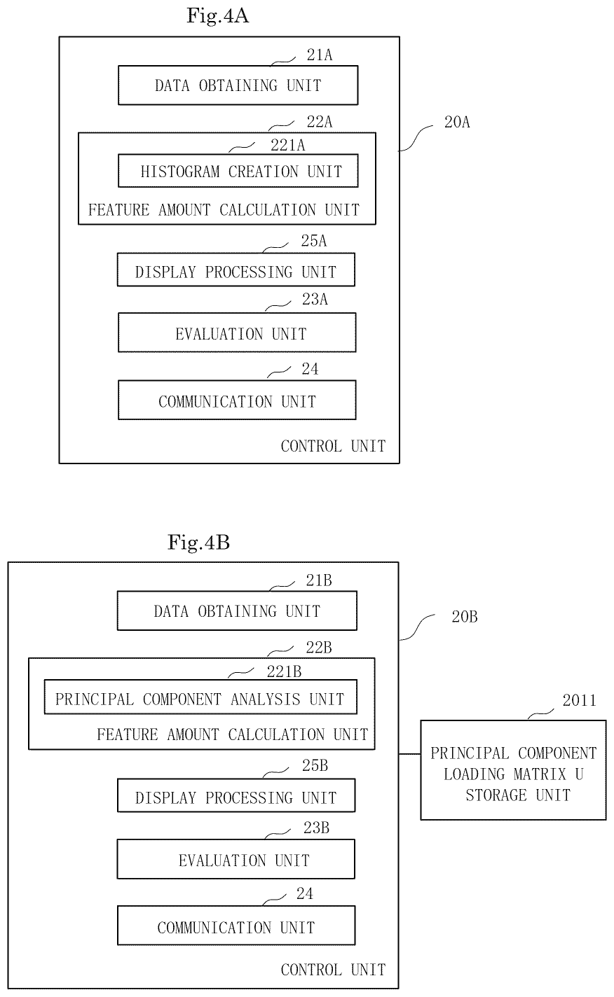

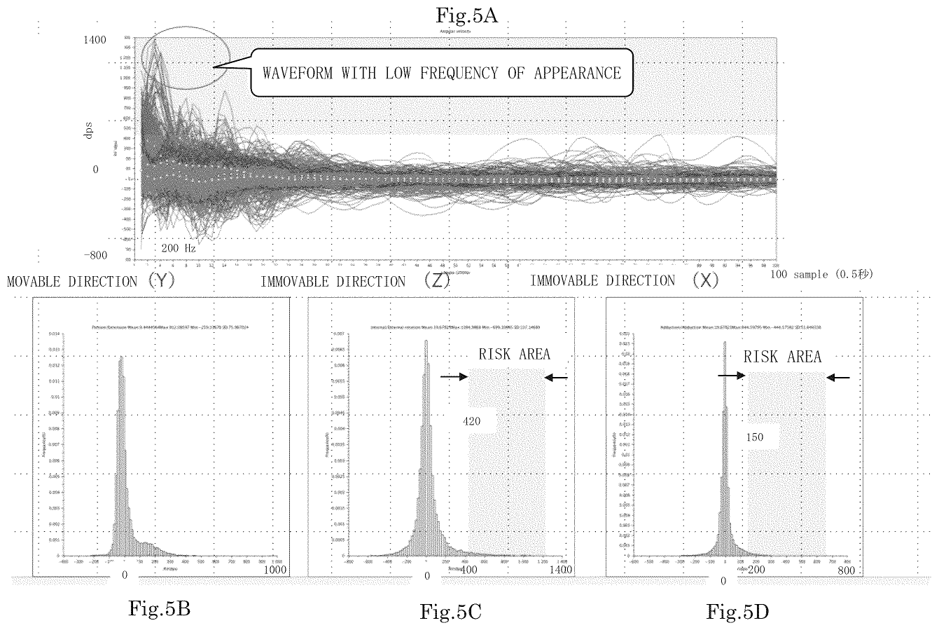

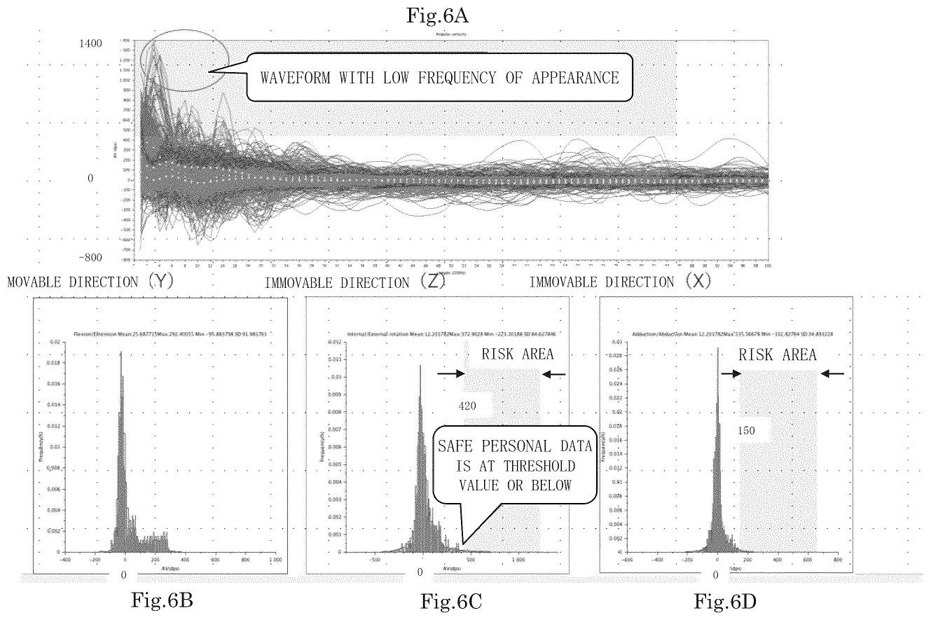

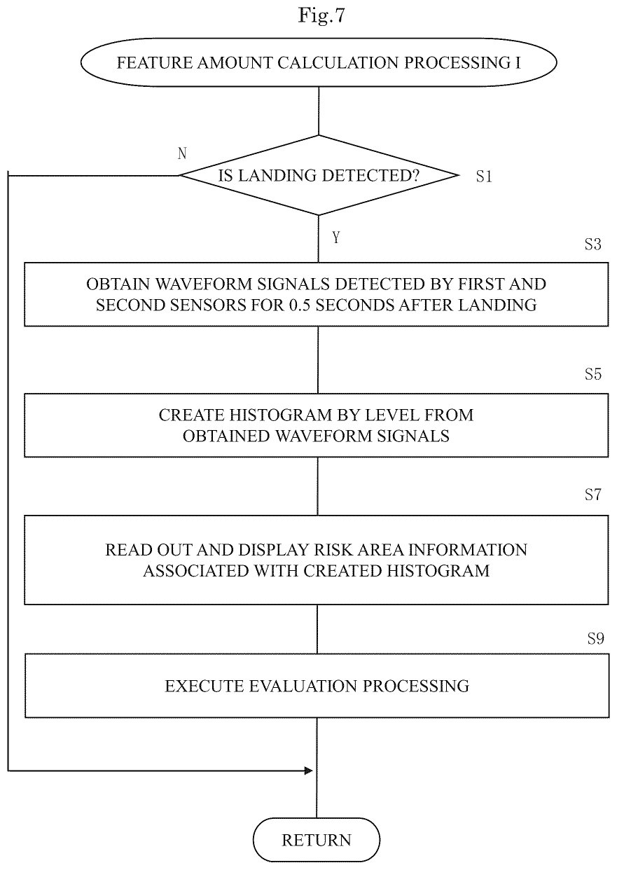

Next, the first embodiment will be described with reference to FIG. 4A, and FIG. 5A to FIG. 5D, FIG. 6A to FIG. 6D and FIG. 7. A control unit 20A includes a data obtaining unit 21A, a feature amount calculation unit 22A, an evaluation unit 23A, a communication unit 24, and a display processing unit 25A. The first embodiment employs a detection signal of the first sensor 31 and the second sensor 32. In other words, the data obtaining unit 21A obtains the detection signal about the joint axis (the w axis (the Z axis) and the u axis (the X axis)) of which the range of motion is limited, among the joint axes.

The feature amount calculation unit 22A includes a histogram creation unit 221A. It is to be noted that the display processing unit 25A displays the creation result on the display unit 202. The histogram creation unit 221A captures a signal sampled with a predetermined period (200 Hz, for example) being a detection signal from the first sensor 31 and the second sensor 32, only for a predetermined time (100 samples: 0.5 seconds, for example). The histogram creation unit 221A extracts detection sampling data from each of the first sensor 31 and the second sensor 32 for each predetermined intensity width, and creates a histogram.

FIG. 5A shows a state in which time is set on the horizontal axis, and intensity, herein, an angular velocity (degree/second) is set on the vertical axis, and the detection signals from the first sensor 31 and the second sensor 32 are mixed. As shown in FIG. 5A, the output of the second sensor 32 has a throughout low level of sway (a dark part on a low-level side) whereas the output of the first sensor 31 is high immediately after a landing (generating a large amount of sway) and gradually decreases over time (a light part on a high-level side). The histogram of FIGS. 5C and 5D indicates this state. The histogram indicates the angular velocity (degree/second) on the horizontal axis, and the number of appearances on the vertical axis.

From data of a large number of subjects, it is recognized that the distribution is close to normal and the variance is small about the joint axis (the w axis (the Z axis)) of which the range of motion is limited, among the joint axes, and that the distribution is close to normal and the level and variance are also small about the joint axis (the u axis (the X axis)) of which the range of motion is limited.

In addition, a risk area shown in FIGS. 5C and 5D is a threshold value of 420 or more (degree/second) about the joint axis (the w axis (the Z axis)), and is a threshold value of 150 or more (degree/second) about the joint axis (the u axis (the X axis)). The threshold value determines the good or bad evaluation of the quality of joint movement, and is evaluated by the amount of a portion exceeding the threshold value, or an exceeding level, or the like. The subject of FIG. 5A to FIG. 5D, in FIG. 5C, accounts for a relatively high percentage of a high angular velocity component exceeding the threshold value, is forecasted to be at high risk of occurrence of injury or disorder, and is evaluated that the quality of movement of the knee joint is not good. On the other hand, in the subject shown in FIG. 6A to FIG. 6D, as shown in FIG. 6C, the high angular velocity component exceeding the threshold value is not a so high percentage, the high angular velocity component itself is rarely seen, so that the quality of movement of the knee joint is evaluated as good or normal. In a period of time immediately after the landing, the frequency of appearance of the high-level waveform of the first sensor 31 is low and variation among individuals is large, which can be the feature amount.

It is to be noted that FIG. 5B and FIG. 6B show, for reference, a histogram based on the detection result from a not-shown sensor integrally provided in the inertial sensor unit 30 that detects the angular velocity about the joint axis (the v axis (the Y axis)), that is, about the joint axis having a movable axis, and by presenting these as necessary, it is possible to evaluate the speed, smoothness, and quality of repeatability of movement of the joint axis with a range of motion.

In this way, at least FIGS. 5C and 5D are displayed on the display unit 202 by the display processing unit 25A, and thus the histogram of the subject and the risk area are shown together, so that the quality of joint movement is able to be easily checked from displayed content. In addition, the evaluation unit 23A makes it possible to quickly perform quality evaluation such as how much of the histogram exceeds the threshold value. Moreover, the result is immediately fed back to the notification unit 36 through the communication units 24 and 35, so that the subject can also know the evaluation results in substantially real time.

FIG. 7 is a flow chart illustrating a procedure of feature amount calculation processing I. First, the joint evaluation apparatus 10 is activated, the inertial sensor unit 30 is made into an operating state, and a time signal detected by the first, the second, and the fourth sensors 31, 32, and 34 is sent toward the control unit 20.

In this state, the data obtaining unit 21 determines whether the acceleration detected by the fourth sensor 34 is 7G or more (Step S1), and, when the acceleration does not reach 7G, returns, and, when the acceleration reaches 7G, determines that the subject has landed on the ground.

Subsequently, the waveform signals detected with a predetermined sampling period by the first and second sensors 31 and 32, only for 0.5 seconds from this landing time point is obtained by the data obtaining unit 21 as new data (Step S3), and is stored in the storage unit 201. Then, when the signals for 0.5 seconds are obtained, a histogram for each level is created from the obtained waveform data by the histogram creation unit 221A (Step S5). Subsequently, risk area information being accumulation data is read out from the storage unit 201 and displayed on the display unit 202 (Step S7), while being associated with the created histogram, as shown in FIGS. 5C and 5D, for example. Subsequently, from the risk area and the created histogram, the good or bad of the created histogram, that is, the good or bad of the quality of joint movement is evaluated based on a degree of high level and a ratio on the high-level side (Step S9). The evaluation processing further immediately sends the evaluation results toward the inertial sensor unit 30, for example, and is notified by the notification unit 36.

Next, the second embodiment will be described with reference to FIG. 4B and FIG. 8A to FIG. 10. A control unit 20B includes a data obtaining unit 21B, a feature amount calculation unit 22B, an evaluation unit 23B, a communication unit 24, and a display processing unit 25B. Furthermore, a storage unit 2011 that stores a principal component loading matrix U is provided. The second embodiment employs an acceleration signal detected by the third sensor 33. In other words, the data obtaining unit 21B obtains the acceleration signal in a direction of the joint axis (the v axis (the Y axis)) of which the range of motion is limited, among the joint axes.

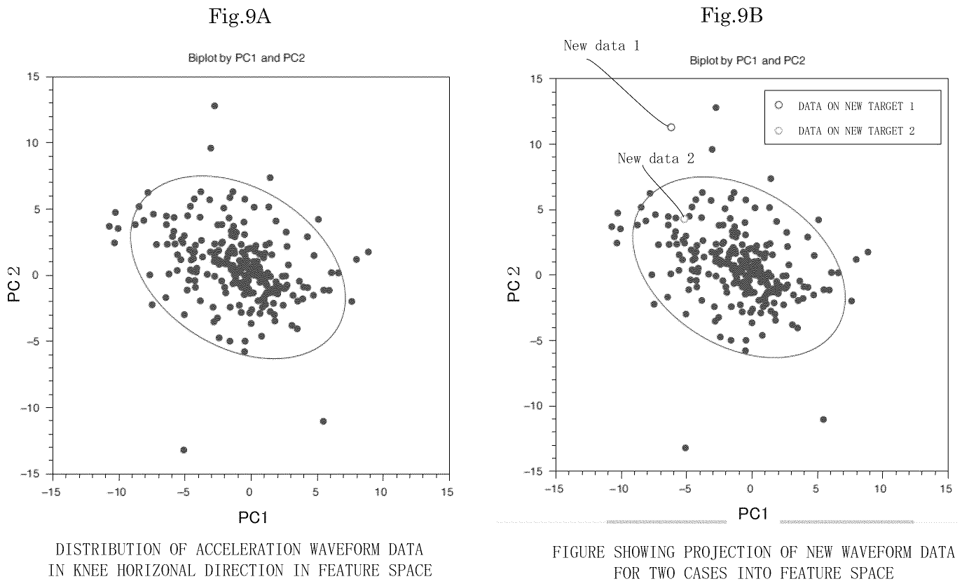

The feature amount calculation unit 22B includes a principal component analysis unit 221B. It is to be noted that the display processing unit 25B displays the analysis result on the display unit 202. The principal component analysis unit 221B captures a signal sampled with a predetermined period (200 Hz, for example) being a detection signal from the third sensor 33, as a waveform signal only for a predetermined time (20 samples: 0.1 seconds, for example). The principal component analysis unit 221B applies the principal component loading matrix U obtained in advance to the captured waveform signal and converts the signal into a feature amount in a principal component space, as described below. In addition, the feature amount of the subject of this time is displayed together (plotted) on an accumulated data distribution map (see FIG. 9A and 9B) by the display processing unit 25B, so that the detailed state of the quality of joint movement is able to be easily recognized. Moreover, the evaluation unit 23B sets up a threshold value to determine the good or bad of the quality of joint movement on the principal component space, and the display processing unit 25B displays together a figure to show the threshold value. The threshold value is displayed as a 95% confidence ellipse being a range including 95% of a plurality of subjects.

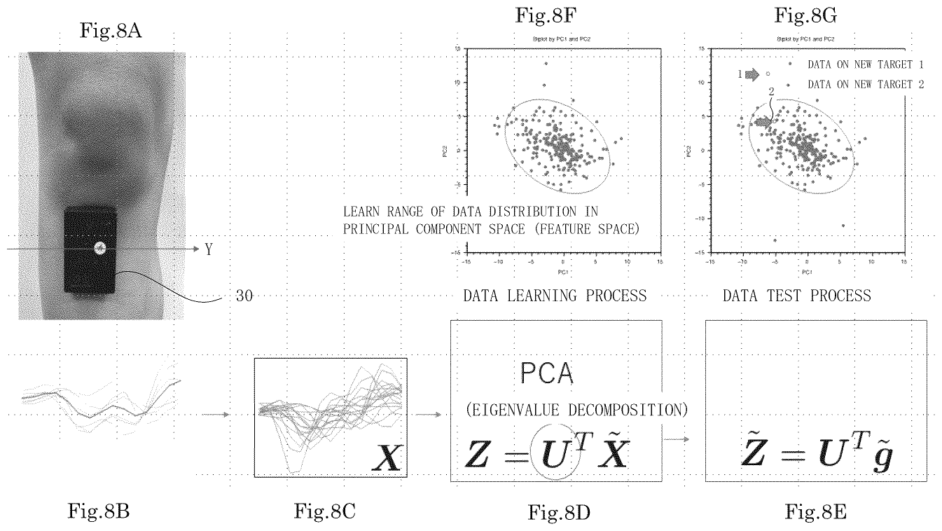

Subsequently, the creation procedure and application procedure of the principal component loading matrix U will be described by use of FIG. 8A to FIG. 8G. After the landing of drop was detected, the acceleration of a tibial tuberosity portion of a landing leg when a subject maintained a stationary standing position for 5 seconds was measured, two types of acceleration of the rapid acceleration in an inward direction early after the landing at that time (a predetermined time after the landing, for example, 0.1 seconds) and the acceleration that repeats fine acceleration and deceleration that occurs during a delayed period of time after the landing were PCA (Principal Component Analysis) performed as data with a high degree of variation, and, furthermore, the individual characteristics of knee joint sway were considered from these principal component plots.

First, the waveform data (see FIG. 8B) including M samples obtained from a subject i among subjects of N persons (300 landings, for example) is represented as in equation (1).

xᵢ={x_{i,j}}ⱼ₌₁^{M} [Mathematical 1]

Then, the data matrix in which the waveform data for the N subjects is arranged in a row is as shown in equation (2) (see FIG. 8C).

$$\begin{matrix}[ Mathematical 2 ] & \\ X = {[ \begin{matrix}{x}_{1 , 1} & {x}_{1 , 2} & … & {x}_{1 , M} \\ {x}_{2 , 1} & {x}_{2 , 2} & … & {x}_{3 , M} \\ ⋮ & ⋮ & ⋱ & ⋮ \\ {x}_{N , 1} & {x}_{N , 2} & … & {x}_{N , M}\end{matrix} ]}_{N \times M} & ( 2 )\end{matrix}$$

Next, equation (3) is obtained as a standardized waveform data matrix standardized for each column (in a time-axis direction) of the data matrix X.

$$\begin{matrix}[ Mathematical 3 ] & \\ \tilde{X} = {\{ \frac{{x}_{i , j} - {\overline{x}}_{j}}{{σ}_{j}} \}}_{N \times M} , ( j = 1 , 2 , … , M ) & ( 3 )\end{matrix}$$

It is to be noted that a bar xⱼ indicates the average value of a column j and σⱼ of indicates the standard deviation of the column j. The principal component analysis performs singular value decomposition on the variance-covariance matrix of the standardized waveform data matrix, as in equation (4), and calculates an eigenvalue and a corresponding eigenvector.

$$\begin{matrix}[ Mathematical 4 ] & \\ C = \frac{{\tilde{X}}^{T} \times \tilde{X}}{N} & ( 4 )\end{matrix}$$

It is to be noted that a superscript T represents a transposition operation of a matrix. An eigenvector matrix including the eigenvector is represented as in equation (5).

$$\begin{matrix}[ Mathematical 5 ] & \\ U = {[ \begin{matrix}{u}_{1 , 1} & {u}_{1 , 2} & … & {u}_{1 , M} \\ {u}_{2 , 1} & {u}_{2 , 2} & … & {u}_{3 , M} \\ ⋮ & ⋮ & ⋱ & ⋮ \\ {u}_{M , 1} & {u}_{M , 2} & … & {u}_{M , M}\end{matrix} ]}_{M \times M} & ( 5 )\end{matrix}$$

Furthermore, the principal component loading matrix U that serves to linearly transform a vector X being the standardized waveform data matrix into a representation Z in a feature space as in equation (6) is obtained (see FIG. 8D).

X=U^{T}\tilde{X} [Mathematical 6]

In a biplot (Biplot) diagram of the principal component scores for the N subjects including a first principal component (PC1) and a second principal component (PC2), data distribution centered on an origin is obtained (FIG. 8F and FIG. 9A). It is to be noted that the inside of the ellipse shown in these figures shows the 95% confidence ellipse.

Herein, the data of the subject that is closer to the origin represents the feature of the motion of the knee in a horizontal direction that is more commonly presented by everyone. On the other hand, the data that is more away from the origin further deviates the pattern of the motion (the acceleration of the Y axis) of the knee in the horizontal direction from normal, and the data outside the 95% confidence ellipse herein is defined as a motion that increases the burden on the knee.

Next, the linear projection of new waveform data into the feature space, and risk detection (a test process), after the principal component loading matrix U is obtained, will be described with reference to FIGS. 8E to 8G and FIG. 9A and FIG. 9B. Equation (7) checks how much the newly obtained waveform data of the subject from the third sensor 33 deviates from the origin in the feature space.

g={g_{i,j}}ⱼ₌₁^{M} [Mathematical 7]

By use of the average value and standard deviation of a learned waveform data matrix X, standardization is performed by equation (8).

$$\begin{matrix}[ Mathematical 8 ] & \\ \tilde{g} = \{ \frac{{g}_{j} - {\overline{r}}_{j}}{{σ}_{j}} \} . ( j = 1 , 2 , … , M ) & ( 8 )\end{matrix}$$

Subsequently, with the principal component loading matrix U obtained (accumulated) in a learning process, a linear mapping to the feature space is performed by equation (9).

\tilde{Z}=H^{T}\tilde{g} [Mathematical 9]

When the first and second principal components of a newly obtained feature vector Z are drawn on the Biplot in FIG. 9A, a figure such as FIG. 9B is obtained. It is to be noted that, in FIG. 9A and FIG. 9B, the horizontal axis indicates PC1 and the vertical axis indicates PC2. The PC1 is the first principal component, with a contribution rate of, for example, 72.7%, and is characterized by the rapid appearance of a peak value in an inward direction immediately after landing, and the PC2 is the second principal component, with a contribution rate of, for example, 24.3%, and is characterized by the slow appearance of a peak value in the inward direction as well as acceleration/deceleration. It is to be noted that the number of principal components is not limited to two, and may be three or more.

FIG. 9B adds data for two new subjects. New data 1 is outside (an outlier region) the 95% confidence ellipse of the distribution of original data. Therefore, movement deviated from a knee pattern common to a large number of subjects is shown, and movement in the horizontal direction that increases the burden on the knee is exhibited, which is targeted for an alert by the evaluation unit 23. In contrast, new data 2 is within the 95% confidence ellipse of the original data, so that the evaluation unit 23 determines the motion of the knee in the horizontal direction is in a normal range and is not targeted for an alert.

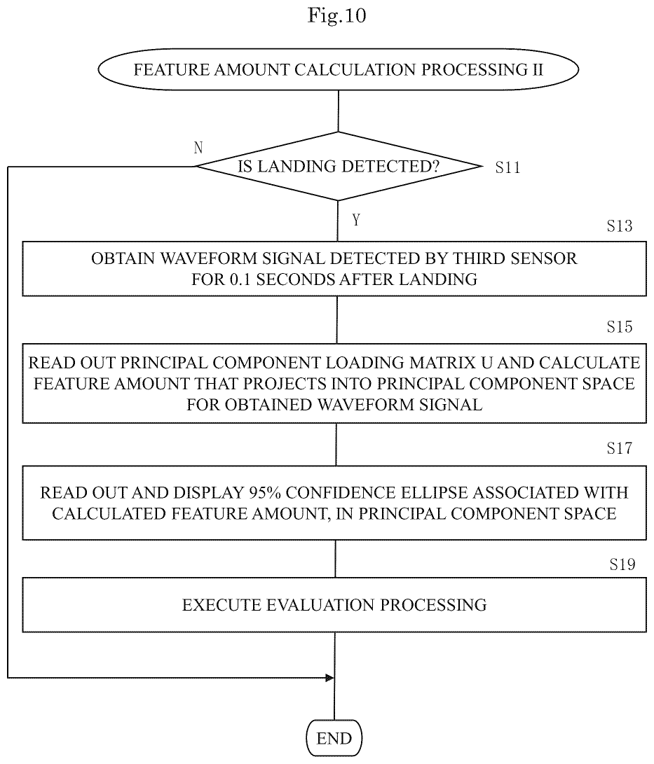

FIG. 10 is a flow chart illustrating a procedure of feature amount calculation processing II. Since Step S11 is the same as Step S1, the description will be omitted.

Subsequently, when the acceleration signal detected by the fourth sensor 34 reaches 7G, the subject is determined to have landed on the ground.

Subsequently, the waveform signal detected by the third sensor 33 with a predetermined sampling period is obtained as new data by the data obtaining unit 21, only for 0.1 seconds from this landing time point (Step S13), and is stored in the storage unit 201. Then, when the waveform signal for 0.1 seconds is obtained, subsequently, the waveform signal obtained by the principal component loading matrix U being the accumulation data read out from the principal component loading matrix U storage unit 2011 is multiplied, and thus the feature amount projected on the principal component space is calculated (Step S15).

Subsequently, 95% confidence ellipse information is read out from the storage unit 201 (or the storage unit 2011) and displayed on the display unit 202, while being associated with the calculated feature amount (Step S17). Subsequently, whether the position of a calculated feature point is within or outside the 95% confidence ellipse being the threshold value is determined, that is, the good or bad of the quality of joint movement is evaluated (Step S19). The evaluation processing may further immediately send the evaluation results toward the inertial sensor unit 30, for example, and may be notified by the notification unit 36.

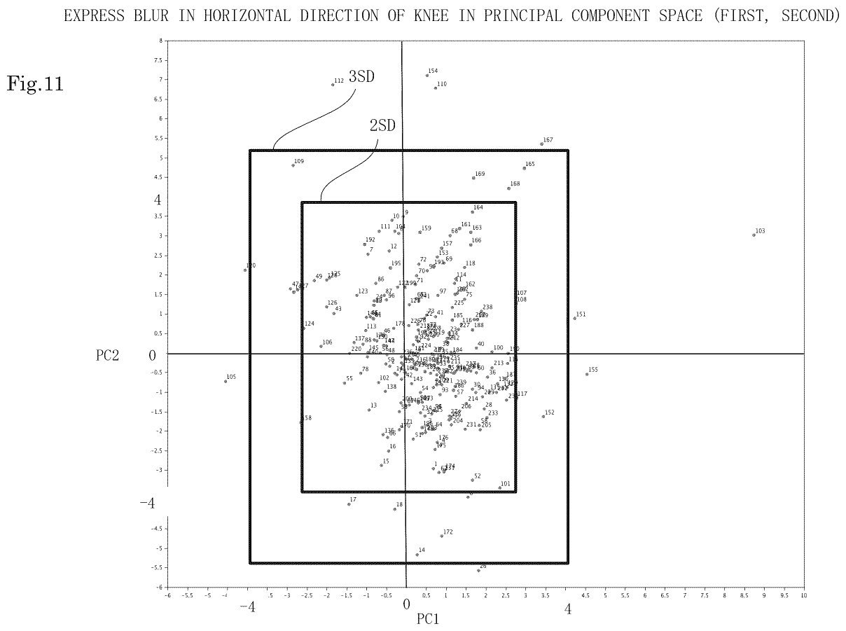

It is to be noted that a threshold region to be displayed in the principal component space may have a quadrangular shape that has been set up to each principal component, as shown in FIG. 11, in addition to an ellipse shape. In the figure, the threshold value is shown as 2SD for a 95% confidence frame being the range including 95% of the plurality of subjects, and as 3SD for a confidence frame including 99% on the outside.

In addition, although, in the present embodiment, one foot is used for the landing (using impact load), both feet may be used for evaluation by bending and stretching of the knee, for example, using load in squat exercise. Moreover, in place of the drop landing, jump landing may be included. Even in such a case, since the principal component loading matrix U according to a load generation mechanism is able to be obtained from the detection information of the motion of a joint of which the range of motion is limited, it is possible to evaluate the quality of joint movement in the load generation mechanism. In addition, the fourth sensor 34 may be configured to be disposed in the inertial sensor unit 30 or on a side of the floor FL, for example.

Moreover, in addition to the knee, even in the elbow, the wrist, the shoulder, or the ankle, the motion of the bone of the joint axis of which the range of motion of joint movement is limited, among the joint axes, is also able to be evaluated by use of the waveform signal.

It is to be noted that, although the sensors 31 and 32 are angular velocity sensors and the sensors 33 and 34 are acceleration sensors, the sensors are not limited to these sensors and may be any type of sensor. It is to be noted that the aspect in which the angular velocity is used, since being less susceptible to the effects of gravity, has an advantage that accuracy improvement is expected.

In addition, the present invention is applicable to various aspects, in addition to forecasting of the risk of occurrence, such as a disorder of a joint. For example, the present invention is able to be used by a coach or a trainer for athlete development through the use of a joint or the like. In addition, the present invention is applicable to movement evaluation and risk watching in rehabilitation using exercise equipment. Furthermore, the present invention is able to be used as a medical diagnostic expert system for joint function quantification.

As described above, the joint evaluation apparatus according to the present invention includes an inertial sensor unit that is attached in a vicinity of a joint with joint axes of the joint to connect bones on both sides parallel to a detection axis and detects motion of a bone of a joint axis of which a range of motion of joint movement is limited, among the joint axes, as a waveform signal, a load detection unit that detects a load applied to the joint, a data obtaining unit that, when detecting generation of the load, obtains the waveform signal to be detected by the inertial sensor unit, in a time direction and an intensity direction, and a feature amount calculation unit that analyzes the waveform signal and calculates a feature amount that evaluates movement quality of the joint.

In addition, the joint evaluation method according to the present invention includes, when a load applied to a joint to connect bones on both sides is detected by a load detection unit, by an inertial sensor unit attached in a vicinity of the joint and having a detection axis parallel to joint axes of the joint, motion of a bone of a joint axis of which a range of motion of joint movement is limited, among the joint axes, as a waveform signal in a time direction and an intensity direction, and analyzing an obtained waveform signal and calculating a feature amount that evaluates movement quality of the joint.

Moreover, the present invention a non-transitory computer readable storage medium storing a program that causes a computer to perform joint evaluation including, when a load applied to a joint to connect bones on both sides is detected by a load detection unit, by an inertial sensor unit attached in a vicinity of the joint and having a detection axis parallel to joint axes of the joint, motion of a bone of a joint axis of which a range of motion of joint movement is limited, among the joint axes, as a waveform signal in a time direction and an intensity direction, and analyzing an obtained waveform signal and calculating a feature amount that evaluates movement quality of the joint.

According to these inventions, the motion of the bone of the joint axis of which the range of motion of the joint movement is limited, among the joint axes, is detected as a waveform signal based on an anatomical basis, by a mounting type inertial sensor unit. The data obtaining unit, when the generation of the load at time of landing to a floor by one-leg drop is detected, for example, obtains the waveform signal to be detected by the inertial sensor unit in the time direction and the intensity direction. The feature amount calculation unit analyzes an obtained waveform signal and calculates the feature amount that evaluates the movement quality of the joint. In this way, by using the mounting type inertial sensor unit, the motion of the bone in the direction of the joint axis of which the range of motion is limited or rotation of the bone about the joint axis of which the range of motion is limited is detected. Then, by obtaining the feature amount, damage, disorder, or the like of such a joint part is able to be easily and accurately evaluated and forecasted, based on the relationship between postural sway, such as left-right or front-back misalignment, torsion, imbalance, or other instabilities, during movement such as drop landing, and the risk of occurrence of damage or disorder. It is to be noted that the inertial sensor unit is not limited to a mode to detect the motion of all or multiple joint axes and may be a detection signal from one target joint axis, for example. In addition, according to a joint part to be examined or a way to apply a load, a joint axis to be detected may be set up appropriately.

Moreover, the inertial sensor unit preferably integrally includes the load detection unit, the load detection unit is preferably an inertia sensor that has a detection axis parallel to a connection direction of the bones on both sides of the joint and detects a generation time point of the load, and the data obtaining unit preferably starts obtaining the waveform signal from the generation time point of the load. According to this configuration, it is possible to detect a load generation signal by the inertial sensor unit.

In addition, the inertial sensor unit preferably includes first and second sensors that detect an angular velocity about two axes mutually orthogonal to a movable joint axis with the range of motion of the joint movement, and the feature amount calculation unit is preferably a histogram creation unit that creates a histogram to signal intensity from the waveform signal as the feature amount. According to this configuration, the movement status of the joint is able to be detected while the effects in gravity is significantly reduced as quickly as possible. Moreover, by using the histogram for each detection level for evaluation, the behavior of the joint of which the range of motion is limited, when a load is applied, is able to be obtained in a form suitable for determination.

In addition, the inertial sensor unit preferably includes two angular velocity sensors with detection axes about a vertical axis of a lower leg and about a sagittal axis of the lower leg. According to this configuration, anatomically meaningful and reliable behavior information is obtained.

Moreover, the inertial sensor unit is preferably a third sensor that has a detection axis parallel to a movable joint axis with the range of motion of the joint movement and detects acceleration, and the feature amount calculation unit is preferably a principal component analysis unit that performs principal component transformation from the waveform signal and calculates the feature amount. According to this configuration, the movement in a direction parallel to the movable joint axis, that is, the direction of which the range of motion is limited, is detected, and the detection signal is further subjected to the principal component transformation for a required type, which makes it possible to easily perform evaluation.

In addition, the principal component analysis unit preferably transforms the waveform signal into first and second principal components, the first principal component preferably shows rapid early sway toward an inner thigh in a period of time immediately after application of the load, and the second principal component preferably shows presence or absence of a peak value in a late period of time to the application of the load. According to this configuration, the principal component space that focuses on information with a high degree of variation among individuals is able to be created, and determination accuracy is maintained.

Moreover, the present invention is preferably a joint evaluation method characterized in that the joint is a knee joint, and the load is a reaction from a landing surface, the reaction being received by a lower leg during a jump from a predetermined height. According to this, inspection work is simplified.

### REFERENCE SIGNS LIST

10 joint evaluation apparatus
20A, 20B control unit
21, 21A, 21B data obtaining unit
22, 22A, 22B feature amount calculation unit (feature amount calculation unit)
221A histogram creation unit (feature amount calculation unit)
221B principal component analysis unit (feature amount calculation unit)
23, 23A, 23B evaluation unit
30 inertial sensor unit
31 first sensor
32 second sensor
33 third sensor
34 fourth sensor (load detection unit)

1. A joint evaluation apparatus comprising:
an inertial sensor unit that is attached in a vicinity of a joint with joint axes of the joint to connect bones on both sides parallel to a detection axis and detects motion of a bone of a joint axis of which a range of motion of joint movement is limited, among the joint axes, as a waveform signal;
a load detection unit that detects a load applied to the joint;
a data obtaining unit that, when detecting generation of the load, obtains the waveform signal to be detected by the inertial sensor unit, in a time direction and an intensity direction; and
a feature amount calculation unit that analyzes the waveform signal and calculates a feature amount that evaluates movement quality of the joint.

2. The joint evaluation apparatus according to claim 1, wherein:
the inertial sensor unit integrally includes the load detection unit;
the load detection unit is an inertia sensor that has a detection axis parallel to a connection direction of the bones on both sides of the joint and detects a generation time point of the load; and
the data obtaining unit starts obtaining the waveform signal from the generation time point of the load.

3. The joint evaluation apparatus according to claim 1, wherein:
the inertial sensor unit includes first and second sensors that detect an angular velocity about two axes mutually orthogonal to a movable joint axis with the range of motion of the joint movement; and
the feature amount calculation unit is a histogram creation unit that creates a histogram to signal intensity from the waveform signal, as the feature amount.

4. The joint evaluation apparatus according to claim 3, wherein the inertial sensor unit includes two angular velocity sensors with detection axes about a vertical axis of a lower leg and about a sagittal axis of the lower leg.

5. The joint evaluation apparatus according to claim 1, wherein:
the inertial sensor unit is a third sensor that has a detection axis parallel to a movable joint axis with the range of motion of the joint movement and detects acceleration; and
the feature amount calculation unit is a principal component analysis unit that performs principal component transformation from the waveform signal and calculates the feature amount.

6. The joint evaluation apparatus according to claim 5, wherein:
the principal component analysis unit transforms the waveform signal into first and second principal components;
the first principal component shows rapid early sway toward an inner thigh in a period of time immediately after application of the load; and
the second principal component shows presence or absence of a peak value in a late period of time to the application of the load.

7. A joint evaluation method comprising:
when a load applied to a joint to connect bones on both sides is detected by a load detection unit, by an inertial sensor unit attached in a vicinity of the joint and having a detection axis parallel to joint axes of the joint, motion of a bone of a joint axis of which a range of motion of joint movement is limited, among the joint axes, as a waveform signal in a time direction and an intensity direction; and
analyzing an obtained waveform signal and calculating a feature amount that evaluates movement quality of the joint.

8. The joint evaluation method according to claim 7, wherein:
the joint is a knee joint; and
the load is a reaction from a landing surface, the reaction being received by a lower leg during a jump from a predetermined height.

9. A non-transitory computer readable storage medium storing a program that causes a computer to perform joint evaluation comprising:
obtaining means that, when detecting generation of the load, obtains the waveform signal to be detected by the inertial sensor unit, in a time direction and an intensity direction, when a load applied to a joint to connect bones on both sides is detected by a load detection unit, by an inertial sensor unit attached in a vicinity of the joint and having a detection axis parallel to joint axes of the joint, motion of a bone of a joint axis of which a range of motion of joint movement is limited, among the joint axes, as a waveform signal in a time direction and an intensity direction; and
analyzing an obtained waveform signal and calculates calculating a feature amount that evaluates movement quality of the joint.
